(19) United States
(12) Patent Application Publication
(10) Pub. No.: US 2011/0064720 A1
(43) Pub. Date: Mar. 17, 2011

(54) Dietary Supplement Compositions and Methods of Making and Using the Same

(76) Inventor: Daniel Moses Amato, Wellington, FL (US)

(21) Appl. No.: 12/883,601

(22) Filed: Sep. 16, 2010

Related U.S. Application Data
(60) Provisional application No. 61/242,845, filed on Sep. 16, 2009.
(60) Provisional application No. 61/262,135, filed on Nov. 17, 2009.

(51) Int. Cl. A61K 38/48; A61K 31/12; A61K 31/235; A61K 31/14; A61K 31/198; A61K 31/145; A61K 31/519; A61K 31/131; A61K 31/385; A61K 31/205; A61P 21/00; A61P 29/00; A61P 5/26; A61P 5/00; A61P 43/00

(52) U.S. Cl. 424/94.65; 514/679; 514/546; 514/642; 514/561; 514/665; 514/257; 514/663; 514/440; 514/554

(57) ABSTRACT

Dietary supplement compositions are disclosed. Methods of making and using dietary supplement compositions are also disclosed.

### CROSS-REFERENCE TO RELATED APPLICATIONS

This patent application claims the benefit of priority to (i) U.S. provisional patent application Ser. No. 61/242,845 entitled “DIETARY SUPPLEMENT COMPOSITIONS AND METHODS OF MAKING AND USING THE SAME” filed on Sep. 16, 2009, and (ii) U.S. provisional patent application Ser. No. 61/262,135 entitled “DIETARY SUPPLEMENT COMPOSITIONS AND METHODS OF MAKING AND USING THE SAME” filed on Nov. 17, 2009, the subject matter of both of which is incorporated herein in its entirety.

### FIELD OF THE INVENTION

The present invention relates generally to dietary supplement compositions. The present invention further relates to methods of making and using dietary supplement compositions.

### BACKGROUND OF THE INVENTION

There is a need in the art for dietary supplement compositions that provide one or more benefits including, but not limited to, heat shock response stimulation for muscle growth; enhance proper protein folding and apoptosis (i.e., death of defective cells); muscle repair; muscle recuperation from training; recovery from trauma, injury or intense exercise; speed muscle growth; pharmacologically inhibit the transcription factor NF-kappaB to enhance muscle regeneration; reduce inflammation; provide anti-aging benefits; modulate estrogen receptor; increase testosterone; and stimulate increases in hormone levels.

### SUMMARY OF THE INVENTION

The present invention is directed to dietary supplement compositions. The dietary supplement compositions comprise a combination of ingredients resulting in dietary supplement compositions that provide one or more benefits including, but not limited to, heat shock response stimulation for muscle growth; enhance proper protein folding and apoptosis (i.e., death of defective cells); muscle repair; muscle recuperation from training; recovery from trauma, injury or intense exercise; speed muscle growth; pharmacologically inhibit the transcription factor NF-kappaB to enhance muscle regeneration; reduce inflammation; provide anti-aging benefits; modulate estrogen receptor; increase testosterone; and stimulate increases in hormone levels.

Accordingly, the present invention is directed to dietary supplement compositions. In one exemplary embodiment of the present invention, the dietary supplement compositions comprise curcumin or a curcumin derivative. The curcumin derivative may comprise, for example, an esterified curcumin having one or more hydroxyl groups thereon converted to an ester. In other exemplary embodiments of the present invention, the dietary supplement compositions comprise curcumin or a curcumin derivative in combination with resveratrol or a resveratrol ester alone or in combination with one or more additional components.

In a further exemplary embodiment of the present invention, the dietary supplement composition of the present invention comprises (or consists essentially of, or consists of) a mixture of curcumin or a curcumin derivative (e.g., an esterified curcumin having one or more hydroxyl groups thereon converted to an ester), resveratrol or a resveratrol ester, carnitine or a carnitine derivative (e.g., (i) L-carnitine tartrate, (ii) acetyl-L-carnitine, (iii) L-carnitine tartrate and acetyl-L-carnitine, or (iv) L-carnitine fumarate), a protease complex comprising (i) papain, (ii) bromelain, or (iii) papain and bromelain, and ursolic acid or an ursolic acid ester. The dietary supplement composition may further comprise (or consists essentially of, or consists of) one or more additional components.

In another exemplary embodiment of the present invention, the dietary supplement composition of the present invention comprises (or consists essentially of, or consists of) at least one aspartic acid or aspartic acid derivative. Exemplary aspartic acids and aspartic acid derivatives include, but are not limited to, D-aspartic acid, L-aspartic acid, n-methyl-d-aspartic acid, L-aspartic acid methyl ester, D-aspartic acid methyl ester, any other aspartic acid ester, any aspartic acid salt, and any combination thereof. The dietary supplement compositions containing at least one aspartic acid or aspartic acid derivative may further comprise a number of additional composition components including, but not limited to, curcumin or a curcumin derivative (e.g., an esterified curcumin having one or more hydroxyl groups thereon converted to an ester), resveratrol or a resveratrol ester, carnitine or a carnitine derivative (e.g., (i) L-carnitine tartrate, (ii) acetyl-L-carnitine, (iii) L-carnitine tartrate and acetyl-L-carnitine or (iv) L-carnitine fumarate), a protease complex comprising (i) papain, (ii) bromelain, or (iii) papain and bromelain, ursolic acid or an ursolic acid ester, piperine extract, a Fungal protease 60,000 HUT, bromelain, a hydrophilic hypromellose polymer, or any combination thereof. The dietary supplement composition may further comprise (or consists essentially of, or consists of) one or more additional components.

The present invention is also directed to methods of making dietary supplement compositions. In one exemplary embodiment of the present invention, the method of making a dietary supplement composition comprises the step of blending one or more composition components with one another to form the resulting dietary supplement composition, wherein the one or more composition components comprise (or consist essentially of, or consist of) curcumin or a curcumin derivative (e.g., an esterified curcumin having one or more hydroxyl groups thereon converted to an ester) alone or in combination with resveratrol or a resveratrol ester, carnitine or a carnitine derivative (e.g., (i) L-carnitine tartrate, (ii) acetyl-L-carnitine, (iii) L-carnitine tartrate and acetyl-L-carnitine or (iv) L-carnitine fumarate), a protease complex comprising (i) papain, (ii) bromelain, or (iii) papain and bromelain, and ursolic acid or an ursolic acid ester. The one or more composition components may further comprise (or consists essentially of, or consists of) one or more additional components.

In another exemplary embodiment of the present invention, the method of making a dietary supplement composition comprises the step of blending one or more composition components with one another to form the resulting dietary supplement composition, wherein the one or more composition components comprise (or consist essentially of, or consist of) at least one aspartic acid or aspartic acid derivative. The step of blending one or more composition components may further comprise blending at least one aspartic acid or aspartic acid derivative with a number of additional composition components including, but not limited to, curcumin or a curcumin derivative (e.g., an esterified curcumin having one or more hydroxyl groups thereon converted to an ester), resveratrol or a resveratrol ester, carnitine or a carnitine derivative (e.g., (i) L-carnitine tartrate, (ii) acetyl-L-carnitine, (iii) L-carnitine tartrate and acetyl-L-carnitine, or (iv) L-carnitine fumarate), a protease complex comprising (i) papain, (ii) bromelain, or (iii) papain and bromelain, ursolic acid or an ursolic acid ester, piperine extract, a Fungal protease 60,000 HUT, bromelain, a hydrophilic hypromellose polymer, or any combination thereof.

The present invention is further directed to methods of using the disclosed dietary supplement compositions. In one exemplary embodiment of the present invention, the method of using the disclosed dietary supplement composition comprises the step of ingesting the dietary supplement composition, wherein the dietary supplement composition comprises curcumin or a curcumin derivative (e.g., an esterified curcumin having one or more hydroxyl groups thereon converted to an ester) alone or in combination with resveratrol or a resveratrol ester, carnitine or a carnitine derivative (e.g., (i) L-carnitine tartrate, (ii) acetyl-L-carnitine, (iii) L-carnitine tartrate and acetyl-L-carnitine, or (iv) L-carnitine fumarate), a protease complex comprising (i) papain, (ii) bromelain, or (iii) papain and bromelain, and ursolic acid or an ursolic acid ester. The dietary supplement composition may further comprise (or consists essentially of, or consists of) one or more additional components.

In another exemplary embodiment of the present invention, the method of using the disclosed dietary supplement composition comprises the step of ingesting the dietary supplement composition, wherein the dietary supplement composition comprises (or consists essentially of, or consists of) at least one aspartic acid or aspartic acid derivative. The ingested dietary supplement composition may further comprise a number of additional composition components including, but not limited to, curcumin or a curcumin derivative (e.g., an esterified curcumin having one or more hydroxyl groups thereon converted to an ester), resveratrol or a resveratrol ester, carnitine or a carnitine derivative (e.g., (i) L-carnitine tartrate, (ii) acetyl-L-carnitine, (iii) L-carnitine tartrate and acetyl-L-carnitine, or (iv) L-carnitine fumarate), a protease complex comprising (i) papain, (ii) bromelain, or (iii) papain and bromelain, ursolic acid or an ursolic acid ester, piperine extract, a Fungal protease 60,000 HUT, bromelain, a hydrophilic hypromellose polymer, or any combination thereof.

These and other features and advantages of the present invention will become apparent after a review of the following detailed description of the disclosed embodiments and the appended claims.

### DETAILED DESCRIPTION OF THE INVENTION

The present invention is directed to dietary supplement compositions. The present invention is further directed to methods of making and using dietary supplement compositions.

The dietary supplement compositions of the present invention may comprise a number of components. A description of individual components and combinations of individual components is provided below.

### I. Dietary Supplement Composition Components

The dietary supplement compositions of the present invention may comprise one or more of the following components.

A. Curcumin or a Curcumin Derivative

The dietary supplement compositions of the present invention comprise curcumin or a curcumin derivative. Curcumin may have one of two structures shown below:

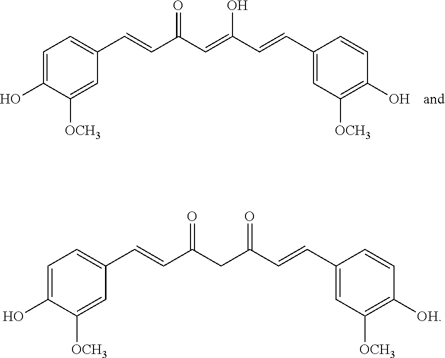

An exemplary curcumin derivative comprises one of the above structures with one of more of the hydroxyl groups thereon converted to an ester, such as a methyl ester or ethyl ester. Typically, the alkyl group used to form the ester comprises from one to eight carbon atoms; however, any alkyl group may be used.

The dietary supplement compositions of the present invention typically comprise greater than 0 to about 7.0 weight percent (wt %) of curcumin or a curcumin derivative based on a total weight of a given dietary supplement composition. In exemplary embodiments, the dietary supplement compositions of the present invention comprise from about 0.5 to about 5.0 wt % (or from about 1.5 to about 4.8 wt %) of curcumin or a curcumin derivative based on a total weight of a given dietary supplement composition.

B. Resveratrol or Resveratrol Ester

The dietary supplement compositions of the present invention may further comprise resveratrol or a resveratrol ester. Resveratrol esters include resveratrol with one of more of the hydroxyl groups thereon converted to an ester, such as a methyl ester or ethyl ester. Typically, the alkyl group used to form the ester comprises from one to eight carbon atoms; however, any alkyl group may be used.

When present, the dietary supplement compositions of the present invention typically comprise greater than 0 to about 1.0 wt % of resveratrol or resveratrol ester based on a total weight of a given dietary supplement composition. In exemplary embodiments, the dietary supplement compositions of the present invention comprise from about 0.0001 to about 0.01 wt % of resveratrol or resveratrol ester based on a total weight of a given dietary supplement composition.

C. Carnitine or Carnitine Derivative

The dietary supplement compositions of the present invention may further comprise carnitine or a carnitine derivative. Suitable carnitine derivatives include, but are not limited to, (i) L-carnitine tartrate, (ii) acetyl-L-carnitine, or (iii) L-carnitine tartrate and acetyl-L-carnitine. When present, the dietary supplement compositions of the present invention typically comprise greater than 0 to about 1.5 wt % of carnitine or carnitine derivative based on a total weight of a given dietary supplement composition. In exemplary embodiments, the dietary supplement compositions of the present invention comprise from about 0.01 to about 0.8 wt % of carnitine or carnitine derivative based on a total weight of a given dietary supplement composition.

D. Protease Complex

The dietary supplement compositions of the present invention may further comprise a protease complex. Suitable protease complexes include, but are not limited to, a protease complex comprising (i) papain, (ii) bromelain, or (iii) papain and bromelain. When present, the dietary supplement compositions of the present invention typically comprise greater than 0 to about 5.0 wt % of a protease complex based on a total weight of a given dietary supplement composition. In exemplary embodiments, the dietary supplement compositions of the present invention comprise from about 1.0 to about 4.0 wt % (or from about 2.0 to about 3.0 wt %) of a protease complex based on a total weight of a given dietary supplement composition.

E. Ursolic Acid or Ursolic Acid Ester

The dietary supplement compositions of the present invention may further comprise ursolic acid or ursolic acid ester. Ursolic acid esters include ursolic acid with one of more of the acid groups thereon converted to an ester, such as a methyl ester or ethyl ester. Typically, the alkyl group used to form the ester comprises from one to eight carbon atoms; however, any alkyl group may be used.

When present, the dietary supplement compositions of the present invention typically comprise greater than 0 to about 1.0 wt % of ursolic acid or ursolic acid ester based on a total weight of a given dietary supplement composition. In exemplary embodiments, the dietary supplement compositions of the present invention comprise from about 0.0001 to about 0.01 wt % of ursolic acid or ursolic acid ester based on a total weight of a given dietary supplement composition.

F. Aspartic Acid or Aspartic Acid Derivative

The dietary supplement compositions of the present invention may further comprise an aspartic acid or an aspartic acid derivative. As used herein, the term “aspartic acid derivative” includes, but is not limited to, any ester of aspartic acid, any salt of aspartic acid, and any complex comprising aspartic acid and a delivery component. Exemplary aspartic acids and aspartic acid derivatives include, but are not limited to, D-aspartic acid, L-aspartic acid, n-methyl-d-aspartic acid, L-aspartic acid methyl ester, D-aspartic acid methyl ester, orotates of aspartic acid, and alpha-ketoglutarates of aspartic acid.

When present, the dietary supplement compositions of the present invention typically comprise greater than 0 to about 10.0 wt % of one or more aspartic acids and/or aspartic acid derivatives based on a total weight of a given dietary supplement composition. In exemplary embodiments, the dietary supplement compositions of the present invention comprise from about 0.0001 to about 5.0 wt % of one or more aspartic acids and/or aspartic acid derivatives based on a total weight of a given dietary supplement composition.

When present, the one or more aspartic acids and/or aspartic acid derivatives provide one or more of the following benefits: enhanced physiological properties via stimulation of the n-methyl-d-aspartate receptor through d-aspartic acid utilization; enhanced antagonistic effects of the NMDA receptor, which stimulates hypothalamus induced hormone increases; targeting of organs so as to stimulate a wide variety of hormonal increases; independent stimulation of hormonal increase on target organs independent of NMDA stimulation; life extension and longevity via youthful hormonal output; bodybuilding and muscle growth and development via anabolic hormone release; sexual performance via increase erectile capacity and libido and testosterone increase; weight loss and mental acuity via hormonal induced pathways; cognitive function via increase of dopamine stimulation by the same pathway; strength increase; endurance enhancement via testosterone, growth hormone, igf-1 and igf-2 increases.

It is believed that the one or more aspartic acids and/or aspartic acid derivatives, through interaction with the n-methyl-d-aspartate receptor and tissue/organ specific pathways, induce hypothalmic, pituitary, ovarie and testi to release one or more of these hormones and neurotransmitters: gonadotropin-releasing hormone (GnRH), testosterone, leutenizing hormone, human growth hormone, dopamine, igf-1, igf-2, melatonin, GHRH (gonadotropic hormone releasing hormone), LHRH (leutinizing hormone releasing hormone), androstenedione, dhea, progesterone, estradiol, prolactin.

It is further believed that the one or more aspartic acids and/or aspartic acid derivatives provide improvement to one or more of the following body functions: erection improvement in men, libido increase in men and women, endurance enhancement for athletic performance, fat loss, strength improvement for athletic performance, increased muscle growth, cognitive enhancement, and body rejuvenating effects for anti-aging.

G. Additional Components

The dietary supplement compositions of the present invention may further comprise one or more of the following additional components as shown in Table 1 below. When present, the dietary supplement compositions of the present invention typically comprise greater than 0 to about 1.0 wt % of a given additional component based on a total weight of a given dietary supplement composition unless otherwise noted in Table 1.

TABLE 1
Exemplary Dietary Supplement Composition Components

| | Label Name/Extract | Typical Weight Percent Based on |
|---|---|---|
| Component | Component | Total Weight of Composition |
| Caffeine Anhydrous | | |
| Theophyline | From Guarana Extract | |
| 1,3-Dimethylamylamine | | |
| Yohimbine HCL | Yohimban-16alpha- | |
| | carboxylic acid, 17beta- | |
| | hydroxy-, methyl ester | |
| | (8CI) | |
| Huperzine (from | | |
| Huperzia serrata) | | |
| L-Theanine | | |
| Vincopoetine | | |
| Glucoronolactone | | |
| L-Tyrosine | | |
| Fucoidan | | |
| L-Arginine | | up to about 75 wt %, typically from |
| | | about 16 to about 66 wt % |
| Di-Arginine Orotate | | up to about 8.0 wt %, typically |
| | | from about 3.0 to about 5.0 wt % |
| Palmatine (Ext. of | o,o-dimethyldemethylene- | |
| Phellodendron) | berberine | |
| L-Arginine Ethyl Ester | | |
| HCL | | |
| L-Citrulline Malate | | |
| L-Norvaline | | |
| Adenosine 5′- | | |
| Triphosphate Disodium | | |
| Creatine Monohydrate | | up to about 20 wt %, typically from |
| | | about 4.0 to about 15 wt % |
| Beta Alanine | | up to about 8.0 wt %, typically |
| | | from about 2.0 to about 4.0 wt % |
| Di-Creatine Orotate | | up to about 10.0 wt %, typically |
| | | from about 2.0 to about 9.0 wt % |
| Creatine Ethyl Ester | | |
| HCL | | |
| Creatinol-O-Phosphate | | |
| Sodium Bicarbonate | | |
| Trimethylglycine | Trimethylaminoacetate | |
| Capsicasum Extract | CH-19 Sweet | |
| Evodiamine | Isoevodiamine | |
| Gamma-Butyrobetaine | | |
| (GBB) Ethyl Ester HCL | | |
| Choline Bitartate | | up to about 3.0 wt %, typically |
| | | from about 1.0 to about 2.0 wt % |
| Potassium Orotate | | up to about 3.0 wt %, typically |
| | | from about 1.0 to about 3.0 wt % |
| Citicoline | (from stabilized cytidine 5′ | |
| | diphosphocholine) | |
| Piperine Extract | | |
| Roobios Extract | Aspalathin | |
| Component | | |
| Roobios Extract | Nothofagin | |
| Component | | |
| L-Leucine Methyl Ester | (2R)-2-Amino-4-Methyl- | |
| HCL | Pentanoic Acid Methyl | |
| | Ester HCL | |
| L-Leucine | | up to about 20 wt %, typically from |
| | | about 12 to about 15 wt % |
| L-Isoleucine | | up to about 4.0 wt %, typically |
| | | from about 0.1 to about 3.0 wt % |
| L-Valine | | up to about 10 wt %, typically from |
| | | about 0.1 to about 7.0 wt % |
| L-Taurine | | up to about 8.0 wt %, typically |
| | | from about 2.0 to about 4.0 wt % |
| Vanadyl Sulfate | Oxidovanadium (2+) | |
| | Sulfate | |
| Bitter Melon | | up to about 3.0 wt %, typically |
| | | from about 1.0 to about 2.0 wt % |
| Lipoic Acid | Sodium R-lipoate | |
| Cinnamon Extract | | up to about 3.0 wt %, typically |
| | | from about 1.0 to about 2.0 wt % |
| Waxy Maize | High Molecular Weight | up to about 20 wt %, typically from |
| | Glucose Polymer | about 13 to about 15 wt % |
| Glycerol Stearate | | up to about 3.0 wt %, typically |
| | | from about 1.0 to about 2.0 wt % |
| Di-Calcium Phosphate | | |
| Di-Potassium Phosphate | | |
| Di-Sodium Phosphate | | |
| Vitamin B-2 | | |
| Vitamin B-3 | | |
| Vitamin B-6 | | RDA - 1500%¹ |
| Vitamin B-9 | | RDA - 10% |
| Vitamin B-12 | as Cyclocobalum and | RDA - 1500% |
| | Dibencozide | |
| Vitamin D-3 | | 200 IU |
| Citric Acid | | up to about 2.0 wt %, typically |
| | | from about 0.1 to about 1.5 wt % |
| Lemon Lime Flavor | | up to about 2.0 wt %, typically |
| | | from about 0.1 to about 1.5 wt % |
| Acesulfame K | | |
| Sucralose | | |
| Silicon Dioxide | | |
| Alfalfa Powder (for | | |
| color) | | |
| Ferulic Acid | Artichoke Plant Extract | |
| Aesculus | | |
| Hippocastanum Extract | | |
| Salix Alba Extract | | |
| Hydrophilic | Time Release Formulation | up to about 3.0 wt %, typically |
| Hypromellose Polymers | | from about 1.0 to about 2.5 wt % |
| Cobalt Orotate | Other forms of Cobalt | up to about 3.0 wt %, typically |
| | may be used | from about 1.0 to about 2.5 wt % |
| Echinacha Purpure | | |
| Extract | | |
| Portulaca Oleracea | | |
| Extract | | |
| Arachadonic Acid | | |
| Sclareolide | Clary Sage Extract | |
| Forskolin | Forskolin (Coleus | |
| | Forskohlii Extract) | |
| Quercetin | | |
| Magnesium Orotate | | |
| Vanadyl Sulfate | Oxidovanadium (2+) | |
| | Sulfate | |
| Berberine HCL | | |
| Riboflavin | | 250% RDA |
| Befiomatine | | |
| Fursultiamine | | |
| Dibencozide | | |
| Vasofolate | | |
| (methyltetrahydrofolate) | | |
| Folate | | up to about 12.0 wt %, typically |
| | | from about 8.0 to about 10 wt % |
| L-Glutamine | | up to about 30 wt %, typically from |
| | | about 24 to about 28 wt % |
| Glutamine AKG | | |
| Glutamine Nitrate | | |
| Glutamine Ethyl Ester | | |
| HCL | | |
| L-Lysine | | up to about 10 wt %, typically from |
| | | about 5.0 to about 7.0 wt % |
| L-Phenylalanine | | up to about 10 wt %, typically from |
| | | about 5.0 to about 7.0 wt % |
| L-Threonine | | up to about 6.0 wt %, typically |
| | | from about 3.0 to about 4.5 wt % |
| L-Histidine | | up to about 5.0 wt %, typically |
| | | from about 2.0 to about 4.0 wt % |
| L-Methionine | | up to about 4.0 wt %, typically |
| | | from about 2.0 to about 3.0 wt % |
| L-Proline | | up to about 2.0 wt %, typically |
| | | from about 0.5 to about 1.5 wt % |
| L-Tryptophan | | up to about 2.0 wt %, typically |
| | | from about 0.5 to about 1.5 wt % |
| Bacopa Monnieri | | |
| Extract (L-DOPA) | | |
| GABA | | up to about 2.0 wt %, typically |
| | | from about 0.5 to about 1.5 wt % |
| GHRP-2 | | |
| L-Glycine | | |
| Phosphoserine | | |
| Ginko Biloba Extract | | |
| Ginseng Extract | | |
| Rhodiola Rosea Extract | | |
| Beta-hydroxy-beta- | | up to about 3.0 wt %, typically |
| methylbutyrate (HMB) | | from about 1.0 to about 2.5 wt % |
| L-Leucine nitrate | | |
| L-isoleucine nitrate | | |
| L-Valine nitrate | | |
| Orange flavor #30001 | | up to about 8.0 wt %, typically |
| | | from about 1.0 to about 5.0 wt % |
| Fungal protease | | up to about 3.0 wt %, typically |
| 60,000 HUT | | from about 1.0 to about 2.5 wt % |
| bromelain | | up to about 3.0 wt %, typically |
| | | from about 1.0 to about 2.5 wt % |
| Glycerol monostearate | | up to about 15.0 wt %, typically |
| | | from about 1.0 to about 13 wt % |
| aspartame | | |
| Phosphatadyl serine | | up to about 3.0 wt %, typically |
| 20% | | from about 1.0 to about 2.5 wt % |
| Thiamin (Vitamin B-1) | | |
| Folic Acid | | |
| Biotin | | |
| Pantothenic Acid | | up to about 3.0 wt %, typically |
| | | from about 1.0 to about 2.5 wt % |
| Vitamin D-3 (Calciferol) | | |
| Cobalt Chelate | | |
| Portulaca Oleracea | | up to about 3.0 wt %, typically |
| Extract (5% Flavones) | | from about 1.0 to about 2.5 wt % |
| Archadonic Acid | | up to about 3.0 wt %, typically |
| | | from about 1.0 to about 2.5 wt % |
| L-Arginine-alpha- | | up to about 50.0 wt %, typically |
| ketoglutarate | | from about 15.0 to about 45.0 wt % |

¹Up to 1500% of the recommended daily amount.

### II. Methods of Making Dietary Supplement Compositions

The present invention is further directed to methods of making the above-described dietary supplement compositions. In one exemplary embodiment of the present invention, the method of making a given dietary supplement composition comprises the step of mixing the above-described composition components to form a powder. In some exemplary embodiments, the method of making a given dietary supplement composition may further comprise one or more of the following steps: weighing one or more composition components prior to mixing; compressing the mixture into tablets using conventional tablet forming techniques; packaging the dietary supplement composition in a container (e.g., a non-pressurized bottle, a bulk plastic container, etc.); providing an amount of dietary supplement composition (e.g., powder or tablet or capsule) as a single serving in a single unit of packaging material; providing on the packaging material, for example, via printed information, a description of a single serving; providing a scoop for powder dietary supplement composition, wherein the scoop measures a single serving of dietary supplement composition; and offering for sale the dietary supplement composition.

In some exemplary embodiments of the present invention, the method of making a dietary supplement composition may comprise specific formulation of ingredients to provide one or more specific functions/benefits. For example, the following combinations of ingredients may be formulated to achieve a particular function/benefit for the resulting composition:

Air-Strike Adreno/Neuro Stimulant Blend:

Caffeine Anhydrous, 1,3-Dimethylamylamine, Theophylline (From Guarana Extract), 1-Oxo-5Beta, 6Beta-Epoxy-Witha-2-Ene-27-Ethoxy-Olide (From Withania Somnifera Extract), Yohimban-16Alpha-Carboxylic Acid, 17Beta-Hydroxy-, and Methyl Ester (8CI)

Smart Munition Nootropic/Cognitive Enhancement System:

Oxiracetam, Huperzine, L-Theanine, Vincopoetine, L-Alpha Glycerylphosphorylcholine, L-Tyrosine, and Acetyl-L-Carnitine Arginate Dihydrochloride

Nitric Oxide Plasma Scorch Technology:

Arginine-O,O-Dimethyldemethyleneberberine Dual Matrix, Fucoidin-Di-Arginine Orotate Inter-Fusion, L-Arginine Nitrate, L-Arginine Ethyl Ester HCL, 3b-Hydroxy-Urs-12-En-28-Oic Acid, L-Citrulline Malate, and L-Norvaline

Advanced Multiphase ATP/Volume Detonation Performance Matrix:

Creatine Monohydrate-Beta Alanine Inter-Fusion, Di-Creatine Orotate, Creatine Ethyl Ester HCL, Creatinol-O-Phosphate, Trimethylglycine, and Sodium Bicarbonate

Core Heat Stress Amplification Fusion:

CH-19 Sweet, Isoevodiamine, and Rutaecarpine

Heat Shock Protein Trigger/Hermetic Agents:

1,9-Bis(4-hydroxy-3-methoxyphenyl)-2,7-nonadiene-4,6-dione

Heat Shock Pathway/Vitagene Encoding Matrix:

5-[2-(4-hydroxyphenyl)ethenyl]benzene-1,3-diol, L-Carnitine Tartrate, L-Carnitine Acetyl Ester, Caffeic Acid 3-Methyl Ether, and Caffeic Acid Phenethyl Ester

Heat Shock Protein Enzymatic Refueling Composite:

Fermented Papaya Extract (Papain), Bromelain, (Protease ph 4.5), (Protease ph 7.0), (Protease ph 3.0) Actinidin, Ficain, and Serratiopeptidase

(FADA-VO2) Fatty Acid Dumping Agent [b-oxidation] VO2-Max Accelerator:

Gamma-Butyrobetaine (GBB) Ethyl Ester HCL, L-Carnitine Fumarate, Choline Bitartrate, and Hippophae Rhamnoides L. Extract

3X-Aminobolic BCAA Matrix:

Aspalathin-Leucine, Nothofagin-L-Isoleucine, and Xanthaurine-L-Valine

(PICIA) Pro-Insulin Cell Infusion Agent:

L-Taurine, Oxidovanadium(2+) Sulfate, Berberine HCL, Bitter Melon, Sodium R-Lipoate, and Cinnamon Extract

PXM-5 Plasma Xpansion Matrix:

Waxy Maize (High Molecular Weight Branched Chain Glucose Polymer)

GPLA-3 Phospho-Glycero Loading Agent:

Di-Calcium Phosphate, Di-Potassium Phosphate, Di-Sodium Phosphate, and Glycerol Stearate

(ESA) EPO Stimulating Matrix:

Cobalt Orotate, Portulaca Oleracea Extract (5% Flavones), (Echinacea Purpurea (whole plant) Echinacea angustifolia (root)) (4% Polyphenols), and Icosa-5,8,11,14-Tetraenoic Acid (Archadonic Acid)

(PVEVA) Vascular System Elasticity/Viscosity Agent:

Salicin (Salix Alba Extract), Nattokinase (Protease Enzyme), Aescin (Aesculus Hippocastanum Extract), and EDTA (Ethylenediaminetetraacetic Acid)

(SSEC) Synergistic (cAMP) Stimulating EPO/Cofactor:

7-beta-Acetoxy-8,13-epoxy-1-alpha,6-beta,9-alpha-trihydroxylabd-14-en-11-one (Coleus Forskohlii Extract), and Decahydro-3a,6,6,9a-tetramethylnaphtho(2,1-b)furan-2(1H)-one (Clary Sage Extract)

VO2-MAX Amplifying Matrix:

2-(3,4-dihydroxyphenyl)-3,5,7-trihydroxy-chromen-4-one (Quercetin), Magnesium Orotate, and Potassium Orotate

(TRB) Time Release Blend:

Hydrophilic Hypromellose Polymers

Heat Shock Pathway/Vitagene Encoding Matrix:

5-[2-(4-hydroxyphenyl)ethenyl]benzene-1,3-diol, L-Carnitine Tartrate, L-Carnitine Acetyl Ester, and Caffeic Acid 3-Methyl Ether

Glutamine Aminobolic Cell-Swelling/HSP Trigger:

L-Glutamine (2-Amino-4-Carbamoyl-Butanoic Acid), Glutamine AKG, Glutamine Ethyl Ester HCL, and Glutamine Nitrate

8× Essential Amino Acid Matrix:

L-Leucine, L-Isoleucine, L-Valine, L-Lysine, L-Phenylalanine, L-Threonine, L-Histidine, and L-Methionine,

HGH Megaton 2-IU Switch:

L-Dopa (2S)-2-Amino-3-(3,4-Dihydroxyphenyl) Propanoic Acid (Bacopa Monnieri Extract), GABA (Gama Amino Butyric Acid), GHRP-2 (Growth Hormone Releasing Peptide-2), and L-Glycine

C-SHOCK Adaptogenic/Anti-Cortisol Recovery Agent:

Phosphoserine, Ginkgolide B (Gingko Biloba Extract), Rosavin, Salidrosides (Rhodiola Rosea Extract), and Ginsenosides Rg3 and Rb1 (Siberian Ginseng Extract)

(PICIA) Pro-Insulin Cell Infusion Agent:

L-Taurine, Oxidovanadium(2+) Sulfate, Berberine HCL, Bitter Melon, Sodium R-lipoate, and Cinnamon Extract

It should be noted that the dietary supplement compositions of the present invention may comprise (consist essentially of, or consist of) any combination of the disclosed composition components. Further, many of the disclosed composition components are optional for a given dietary supplement composition, and in some embodiments, specifically left out of a given dietary supplement composition. For example, some of the dietary supplement compositions of the present invention may be specifically formulated to not contain one or more of the following disclosed composition components: caffeine, creatine or creatine derivatives (e.g., creatine monohydrate, di-creatine orotate, creatine ethyl ester HCL, and creatinol-o-phosphate), and artificial dyes.

Any of the disclosed dietary supplement compositions of the present invention may be formulated to have a desired maximum level of carbohydrates therein. For example, in some embodiments, the dietary supplement composition of the present invention comprises (or consists of) less than about 10 grams (g) of carbohydrates per serving (or dose). In other embodiments, the dietary supplement composition of the present invention comprises (or consists of) less than about 5.0 g (or less than 3.0 g, or less than 1.0 g) of carbohydrates per serving (or dose).

As shown in examples below, some of the dietary supplement compositions of the present invention are specifically formulated to contain one or more of the following specific disclosed composition components: curcumin or a curcumin derivative (e.g., an esterified curcumin having one or more hydroxyl groups thereon converted to an ester); resveratrol or a resveratrol ester (e.g., resveratrol triacetate ester); carnitine or a carnitine derivative (e.g., (i) L-carnitine tartrate, (ii) acetyl-L-carnitine, (iii) L-carnitine tartrate and acetyl-L-carnitine or (iv) L-carnitine fumarate); a protease complex comprising (i) papain, (ii) bromelain, or (iii) papain and bromelain; ursolic acid or an ursolic acid ester; 1,3-dimethylamylamine; d-aspartic acid; n-methyl-d-aspartate; vitamin D; arginine or an arginine derivative (e.g., arginine alpha ketoglutarate); and S-adenosyl methionine (SAMe).

### III. Methods of Using Dietary Supplement Compositions

The present invention is further directed to methods of using the above-described dietary supplement compositions. In one exemplary embodiment of the present invention, the method of using a dietary supplement composition comprises the step of ingesting the dietary supplement composition of the present invention. The dietary supplement composition may be ingested via one of the following forms: oral capsule, powder drink, tablet, spray, and liquid drink.

In other embodiments, a dietary supplement composition may be applied to a user via one or more of the following methods of delivery: transdermal cream, intra mucous membrane suppository, nasal spray, and oral strip. In some exemplary embodiments of the present invention, the method of using a dietary supplement composition comprises one or more of the following steps: mixing the dietary supplement composition with a liquid (e.g., water, soda, coffee); and adding the dietary supplement to food (e.g., baked goods, yogurt, fruit).

In other embodiments, the present invention is directed to methods of providing one or more benefits including, but not limited to, heat shock response stimulation for muscle growth; enhance proper protein folding and apoptosis (i.e., death of defective cells); muscle repair; muscle recuperation from training; recovery from trauma, injury or intense exercise; speed muscle growth; pharmacologically inhibit the transcription factor NF-kappaB to enhance muscle regeneration; reduce inflammation; provide anti-aging benefits; modulate estrogen receptor; increase testosterone; and stimulate increases in hormone levels via ingestion of one or more of the disclosed dietary supplement compositions described herein.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

It should be noted that in the following examples the term “up to 25 mg” (or any other amount) is used to indicate that a given component is present in an amount of greater than 0 up to 25 mg (or any other amount listed with the phrase “up to”).

### Example 1

### Preparation of Exemplary Dietary Supplement Compositions

Exemplary dietary supplement compositions of the present invention were prepared by mixing various amounts of the following components shown in Table 2 below. For example, a given exemplary composition may comprise any amount of L-Theanine up to 500 mg (i.e., any amount greater than 0 up to 500 mg), but comprises 105 mg of L-Tyrosine.

TABLE 2
Exemplary Dietary Supplement Compositions

| Component | Label Name/Extract Component | Amount | |
|---|---|---|---|
| Caffeine Anhydrous | | up to 400 | mg |
| Theophyline | From Guarana Extract | up to 100 | mg |
| 1,3-Dimethylamylamine | | up to 200 | mg |
| Yohimbine HCL | Yohimban-16alpha-carboxylic acid, | 2 | mg |
| | 17beta-hydroxy-, methyl ester (8CI) | | |
| Huperzine (from Huperzia | | up to 500 | mg |
| serrata) | | | |
| L-Theanine | | up to 500 | mg |
| Vincopoetine | | up to 500 | mg |
| Glucoronolactone | | up to 500 | mg |
| L-Tyrosine | | 105 | mg |
| Fucoidan | | up to 500 | mg |
| L-Arginine | | 2.5 | g |
| Di-Arginine Orotate | | 500 | mg |
| Palmatine (Ext. of | o,o-dimethyldemethyleneberberine | up to 500 | mg |
| Phellodendron) | | | |
| L-Arginine Ethyl Ester HCL | | up to 500 | mg |
| Ursolic Acid | 3b-Hydroxy-Urs-12-En-28-Oic Acid (as | up to 500 | mg |
| | Danshen extract) | | |
| L-Citrulline Malate | | 100 | mg |
| L-Norvaline | | 100 | mg |
| Adenosine 5′-Triphosphate | | up to 500 | mg |
| Disodium | | | |
| Creatine Monohydrate | | 2 | g |
| Beta Alanine | | 500 | mg |
| Di-Creatine Orotate | | 1 | g |
| Creatine Ethyl Ester HCL | | up to 500 | mg |
| Creatinol-O-Phosphate | | up to 500 | mg |
| Sodium Bicarbonate | | 100 | mg |
| Trimethylglycine | Trimethylaminoacetate | up to 500 | mg |
| Capsicasum Extract | CH-19 Sweet | up to 500 | mg |
| Evodiamine | Isoevodiamine | up to 500 | mg |
| Curcumin (High Potency | 1,9-Bis(4-hydroxy-3-methoxyphenyl)- | 200 | mg |
| Cucurmin Extract) | 2,7-nonadiene-4,6-dione | | |
| Resveratrol (Grape Seed | 5-[2-(4-hydroxyphenyl)ethenyl]benzene- | up to 500 | mg |
| Extract) | 1,3-diol | | |
| L-Carnitine Tartrate | | 50 | mg |
| Acetyl-L-Carnitine | | up to 500 | mg |
| Gamma- | | up to 500 | mg |
| Butyrobetaine(GBB) Ethyl | | | |
| Ester HCL | | | |
| Choline Bitartate | | 200 | mg |
| Potassium Orotate | | 250 | mg |
| Citicoline | (from stabilized cytidine 5′ | up to 500 | mg |
| | diphosphocholine) | | |
| L-Carnitine Fumarate | | 50 | mg |
| Protease Complex | Fermented Papaya Extract (Papain), | 400 | mg |
| | Bromelain, (Protease ph 4.5), (Protease | | |
| | ph 7.0), (Protease ph 3.0) Actinidin, | | |
| | Ficain, Serratiopeptidase | | |
| Piperine Extract | | | |
| Roobios Extract Component | Aspalathin | up to 500 | mg |
| Roobios Extract Component | Nothofagin | up to 500 | mg |
| L-Leucine Methyl Ester | (2R)-2-Amino-4-Methyl-Pentanoic Acid | up to 500 | mg |
| HCL | Methyl Ester HCL | | |
| L-Leucine | | 2000 | mg |
| L-Isoleucine | | up to 500 | mg |
| L-Valine | | up to 500 | mg |
| L-Taurine | | 500 | mg |
| Vanadyl Sulfate | Oxidovanadium (2+) Sulfate | up to 500 | mg |
| Bitter Melon | | 200 | mg |
| Lipoic Acid | Sodium R-lipoate | 20 | mg |
| Cinnamon Extract | | 250 | mg |
| Waxy Maize | High Molecular Weight Glucose Polymer | 2 | g |
| Glycerol Stearate | | 200 | mg |
| Di-Calcium Phosphate | | 100 | mg |
| Di-Potassium Phosphate | | 100 | mg |
| Di-Sodium Phosphate | | 100 | mg |
| Vitamin B-2 | | 25 | mg |
| Vitamin B-3 | | 25 | mg |
| Vitamin B-6 | | RDA - 1500% | |
| Vitamin B-9 | | RDA - 10% | |
| Vitamin B-12 | As Cyclocobalum and Dibencozide | RDA - 1500% | |
| Vitamin D-3 | | 200 | IU |
| Citric Acid | | up to 500 | mg |
| Lemon Lime Flavor | | up to 500 | mg |
| Acesulfame K | | up to 500 | mg |
| Sucralose | | up to 500 | mg |
| Silicon Dioxide | | up to 500 | mg |
| Alfalfa Powder (for color) | | up to 500 | mg |

The resulting compositions had a powder consistency and a fruit punch/orange flavor.

### Example 2

### Preparation of Exemplary Dietary Supplement Compositions

Other exemplary dietary supplement compositions of the present invention were prepared by mixing various amounts of the following components shown in Table 3 below.

TABLE 3
Exemplary Dietary Supplement Compositions

| Component | Label Name/Extract Component | Amount | |
|---|---|---|---|
| Fucoidan | | up to 500 | mg |
| L-Arginine | | 2.8 | g |
| Di-Arginine Orotate | | 200 | mg |
| (Palmatine) Extract of | O,O-Dimethyldemethyleneberberine | up to 500 | mg |
| Phellodendron | | | |
| L-Arginine Ethyl Ester HCL | | up to 500 | mg |
| Ursolic Acid | 3b-Hydroxy-Urs-12-En-28-Oic Acid (as | up to 500 | mg |
| | Danshen extract) | | |
| L-Citrulline Malate | | up to 500 | mg |
| L-Norvaline | | up to 500 | mg |
| Adenosine 5′-Triphosphate | | up to 500 | mg |
| Disodium | | | |
| Creatine Monohydrate | | 200 | mg |
| Beta Alanine | | up to 500 | mg |
| Di-Creatine Orotate | | 100 | mg |
| Creatine Ethyl Ester HCL | | up to 500 | mg |
| Creatinol-O-Phosphate | | up to 500 | mg |
| Trimethylglycine | Trimethylaminoacetate | up to 500 | mg |
| Curcumin (High Potency | 1,9-Bis(4-hydroxy-3-methoxyphenyl)- | 200 | mg |
| Cucurmin Extract) | 2,7-nonadiene-4,6-dione | | |
| Resveratrol (Grape Seed | 5-[2-(4-hydroxyphenyl)ethenyl]benzene- | up to 500 | mg |
| Extract) | 1,3-diol | | |
| L-Carnitine Tartrate | | up to 500 | mg |
| Acetyl-L-Carnitine | | up to 500 | mg |
| Ferulic Acid | Artichoke Plant Extract | up to 500 | mg |
| Protease Complex | Fermented Papaya Extract (Papain), | 25 | mg |
| | Bromelain, (Protease ph 4.5), (Protease | | |
| | ph 7.0), (Protease ph 3.0) Actinidin, | | |
| | Ficain, Serratiopeptidase, Nattokinase | | |
| Aesculus Hippocastanum | | up to 500 | mg |
| Extract | | | |
| Salix Alba Extract | | up to 500 | mg |
| Hydrophilic Hypromellose | Time Release Formulation | 100 | mg |
| Polymers | | | |
| Cobalt Orotate | Other forms of Cobalt may be used | 100 | mcg |
| Echinacha Purpure Extract | | up to 500 | mg |
| Portulaca Oleracea Extract | | 10 | mg |
| Arachadonic Acid | | 10 | mg |
| Sclareolide | Clary Sage Extract | 5 | mg |
| Forskolin | Forskolin (Coleus Forskohlii Extract) | 5 | mg |
| Quercetin | | 5 | mg |
| Potassium Oroate | | 5 | mg |
| Magnesium Orotate | | 5 | mg |
| Vanadyl Sulfate | Oxidovanadium (2+) Sulfate | 3 | mg |
| Berberine HCL | | 5 | mg |
| Bitter Melon | | up to 500 | mg |
| Lipoic Acid | Sodium R-lipoate | 5 | mg |
| Cinnamon Extract | | 10 | mg |
| Vitamin B-2 | as Riboflavin, Benfiomatine and | up to 500 | mg |
| | Fursultiamine | | |
| Vitamin B-3 | Niacin | 30 | mg |
| Vitamin B-6 | (as Pyridoxine HCL And Pyridoxine 5- | 25 | mg |
| | Phosphate) | | |
| Vitamin B-9 | Folate (see below) | 100% | |
| Vitamin B-12 | As Dibencozide and other cofactors | 1500% | |
| Riboflavin | | 250% RDA | |
| Befiomatine | | up to 500 | mg |
| Fursultiamine | | up to 500 | mg |
| Dibencozide | | up to 500 | mg |
| Vasofolate | | up to 500 | mg |
| (methyltetrahydrofolate) | | | |
| Folate | | 400 | mg |

The resulting compositions were formed into caplets and bottled.

### Example 3

### Preparation of Exemplary Dietary Supplement Compositions

Other exemplary dietary supplement compositions of the present invention were prepared by mixing various amounts of the following components shown in Table 4 below.

TABLE 4
Exemplary Dietary Supplement Compositions

| Component | Label Name/Extract Component | Amount | |
|---|---|---|---|
| Creatine Monohydrate | | 2 | g |
| Beta Alanine | | 500 | mg |
| Di-Creatine Orotate | | 1 | g |
| Creatine Ethyl Ester HCL | | up to 500 | mg |
| Creatinol-O-Phosphate | | up to 500 | mg |
| Trimethylglycine | Trimethylaminoacetate | up to 500 | mg |
| Curcumin (High Potency | 1,9-Bis(4-hydroxy-3-methoxyphenyl)- | 200 | mg |
| Cucurmin Extract) | 2,7-nonadiene-4,6-dione | | |
| Resveratrol (Grape Seed | 5-[2-(4-hydroxyphenyl)ethenyl]benzene- | up to 500 | mg |
| Extract) | 1,3-diol | | |
| L-Carnitine Tartrate | | 50 | mg |
| Acetyl-L-Carnitine | | up to 500 | mg |
| Ferusolic Acid | Artichoke Plant Extract | up to 500 | mg |
| Protease Complex | Fermented Papaya Extract (Papain), | 200 | mg |
| | Bromelain, (Protease ph 4.5), (Protease | | |
| | ph 7.0), (Protease ph 3.0) Actinidin, | | |
| | Ficain, Serratiopeptidase, Nattokinase | | |
| L-Glutamine | | 5 | g |
| Glutamine AKG | | up to 500 | mg |
| Glutamine Nitrate | | up to 500 | mg |
| Glutamine Ethyl Ester HCL | | up to 500 | mg |
| L-Leucine | | 2500 | mg |
| L-Isoleucine | | 500 | mg |
| L-Valine | | 1200 | mg |
| L-Lysine | | 1200 | mg |
| L-Phenylalanine | | 1200 | mg |
| L-Threonine | | 800 | mg |
| L-Histidine | | 650 | mg |
| L-Methionine | | 450 | mg |
| L-Proline | | 250 | mg |
| L-Tryptophan | | 250 | mg |
| Bacopa Monnieri Extract | | 50 | mg |
| (L-DOPA) | | | |
| GABA | | 200 | mg |
| GHRP-2 | | up to 500 | mg |
| L-Glycine | | 50 | mg |
| Phosphoserine | | 5 | mg |
| Ginko Biloba Extract | | 50 | mg |
| Ginseng Extract | | 50 | mg |
| Rhodiola Rosea Extract | | 50 | mg |
| L-Taurine | | 500 | mg |
| Vanadyl Sulfate | Oxidovanadium (2+) Sulfate | 3 | mg |
| Bitter Melon | | 200 | mg |
| Lipoic Acid | Sodium R-lipoate | 20 | mg |
| Cinnamon Extract | | 250 | mg |
| Citric Acid | | up to 500 | mg |
| Lemon Lime Flavor | | up to 500 | mg |
| Acesulfame K | | up to 500 | mg |
| Sucralose | | up to 500 | mg |
| Silicon Dioxide | | up to 500 | mg |
| Alfalfa Powder (for color) | | up to 500 | mg |

The resulting compositions had a powder consistency and a fruit punch/orange flavor.

### Example 4

### Preparation of Exemplary Dietary Supplement Compositions

Exemplary dietary supplement compositions of the present invention were prepared by mixing various amounts of the following components shown in Table 5 below. For example, a given exemplary composition may comprise any amount of resveratrol up to 500 mg (i.e., any amount greater than 0 up to 500 mg), but comprises 100 mg of curcumin.

TABLE 5
Exemplary Dietary Supplement Compositions

| Component | Label Name/Extract Component | Amount | |
|---|---|---|---|
| Caffeine Anhydrous | | up to 500 | mg |
| 1,3-Dimethylamylamine | | up to 200 | mg |
| L-Taurine | | up to 4000 | mg |
| Glucoronolactone | | up to 1000 | mg |
| Resveratrol (Grape Seed | 5-[2-(4-hydroxyphenyl)ethenyl]benzene- | up to 500 | mg |
| Extract) 50% | 1,3-diol | | |
| Curcumin (High Potency | 1,9-Bis(4-hydroxy-3-methoxyphenyl)- | 100 | mg |
| Cucurmin Extract) | 2,7-nonadiene-4,6-dione | | |
| resveratrol tri-acetate ester | | 50 | mg |
| curcumin mono-acetate | | up to 50 | mg |
| ester | | | |
| curcumin di-acetate ester | | up to 50 | mg |
| alpha-Lipoic Acid | Sodium R-lipoate | 25 | mg |
| Acetyl-L-Carnitine HCL | | 25 | mg |
| Evodiamine extract 98% | Isoevodiamine | up to 50 | mg |
| Di-Arginine Orotate | | 900 | mg |
| Trimethylglycine | Trimethylaminoacetate | 50 | mg |
| Beta Alanine | | 50 | mg |
| L-aspartic acid | | 500 | mg |
| D-aspartic acid | | up to 500 | mg |
| n-methyl-d-aspartate | | up to 50 | mg |
| D-aspartic acid methyl ester | | up to 50 | mg |
| L-Leucine | | 200 | mg |
| Beta-hydroxy-beta- | | 200 | mg |
| methylbutyrate (HMB) | | | |
| L-Leucine nitrate | | 50 | mg |
| L-isoleucine | | 250 | mg |
| L-isoleucine nitrate | | 50 | mg |
| L-Valine | | 250 | mg |
| L-Valine nitrate | | 50 | mg |
| Piperine Extract 95% | bioperine | 5 | mg |
| Orange flavor #30001 | | 600 | mg |
| Citric Acid | | 800 | mg |
| Acesulfame K | | 50 | mg |
| Fungal protease | | 100 | mg |
| 60,000 HUT | | | |
| bromelain | | 100 | mg |
| Glycerol monostearate | | 1000 | mg |
| sucralose | | 150 | mg |
| aspartame | | 30 | mg |

The resulting compositions had a powder consistency and an orange flavor.

### Example 5

### Preparation of Exemplary Dietary Supplement Compositions

Exemplary dietary supplement compositions of the present invention were prepared by mixing various amounts of the following components shown in Table 6 below. For example, a given exemplary composition may comprise any amount of resveratrol 95% up to 500 mg (i.e., any amount greater than 0 up to 500 mg), but comprises 100 mg of curcumin.

TABLE 6
Exemplary Dietary Supplement Compositions

| Component | Label Name/Extract Component | Amount | |
|---|---|---|---|
| Resveratrol (Grape Seed | 5-[2-(4-hydroxyphenyl)ethenyl]benzene- | up to 500 | mg |
| Extract) 95% | 1,3-diol | | |
| Curcumin (High Potency | 1,9-Bis(4-hydroxy-3-methoxyphenyl)- | 100 | mg |
| Cucurmin Extract) | 2,7-nonadiene-4,6-dione | | |
| resveratrol tri-acetate ester | | up to 50 | mg |
| curcumin mono-acetate | | up to 50 | mg |
| ester | | | |
| curcumin di-acetate ester | | up to 50 | mg |
| alpha-Lipoic Acid | Sodium R-lipoate | 25 | mg |
| Acetyl-L-Carnitine HCL | | 25 | mg |
| L-glutamine | | 1000 | mg |
| L-Arginine HCL | | 1000 | mg |
| L-aspartic acid | | 500 | mg |
| D-aspartic acid | | up to 500 | mg |
| n-methyl-d-aspartate | | up to 50 | mg |
| D-aspartic acid methyl ester | | up to 50 | mg |
| L-Leucine | | 1000 | mg |
| L-isoleucine | | 250 | mg |
| L-Valine | | 250 | mg |
| L-Lysine | | 500 | mg |
| L-Phenylalanine | | 500 | mg |
| L-Threonine | | 500 | mg |
| L-Histidine | | 250 | mg |
| L-Methionine | | 250 | mg |
| Waxy Maize | High Molecular Weight Glucose Polymer | 4000 | mg |
| Piperine Extract 95% | | 5 | mg |
| Lemon lime flavor #30001 | | 600 | mg |
| Citric Acid | | 800 | mg |
| Acesulfame K | | 50 | mg |
| Fungal protease | | 100 | mg |
| 60,000 HUT | | | |
| bromelain | | 100 | mg |
| Glycerol monostearate | | 1000 | mg |
| Phosphatadyl serine 20% | | 100 | mg |
| sucralose | | 150 | mg |
| aspartame | | 30 | mg |

The resulting compositions had a powder consistency and a lemon lime flavor.

### Example 6

### Preparation of Exemplary Dietary Supplement Compositions

Exemplary dietary supplement compositions of the present invention were prepared by mixing various amounts of the following components shown in Table 7 below. For example, a given exemplary composition may comprise any amount of n-methyl-d-aspartate up to 50 mg (i.e., any amount greater than 0 up to 50 mg), but comprises 50 mg of thiamin.

TABLE 7
Exemplary Dietary Supplement Compositions

| Component | Label Name/Extract Component | Amount | |
|---|---|---|---|
| Thiamin (Vitamin B-1) | | 50 | mg |
| Riboflavin (Vitamin B-2) | | 50 | mg |
| Niacin (Vitamin B-3) | | 50 | mg |
| Vitamin B-6 | (as Pyridoxine HCL) | 50 | mg |
| Folic Acid | | 400 | mcg |
| Biotin | | 300 | mcg |
| Pantothenic Acid | | 100 | mg |
| Vitamin D-3 (Calciferol) | | 2000 | iu |
| Cobalt Chelate | | 25 | mcg |
| Portulaca Oleracea Extract | | 100 | mg |
| (5% Flavones) | | | |
| Archadonic Acid | | 100 | mg |
| Resveratrol (Grape Seed | 5-[2-(4- | 50 | mg |
| Extract) 95% | hydroxyphenyl)ethenyl]benzene- | | |
| | 1,3-diol | | |
| Curcumin (High Potency | 1,9-Bis(4-hydroxy-3-methoxyphenyl)- | 100 | mg |
| Cucurmin Extract) | 2,7-nonadiene-4,6-dione | | |
| resveratrol tri-acetate ester | | up to 50 | mg |
| curcumin mono-acetate | | up to 100 | mg |
| ester | | | |
| curcumin di-acetate ester | | up to 100 | mg |
| alpha-Lipoic Acid | Sodium R-lipoate | 25 | mg |
| Acetyl-L-Carnitine HCL | | 25 | mg |
| L-Arginine-alpha- | | 3000 | mg |
| ketoglutarate | | | |
| L-aspartic acid | | 500 | mg |
| D-aspartic acid | | up to 500 | mg |
| n-methyl-d-aspartate | | up to 50 | mg |
| D-aspartic acid methyl ester | | up to 50 | mg |
| Piperine Extract 95% | | 5 | mg |
| Fungal protease | | 100 | mg |
| 60,000 HUT | | | |
| bromelain | | 100 | mg |
| Hydrophilic hypromellose | | 200 | mg |
| polymers | | | |

### Example 7

### Preparation of Exemplary Dietary Supplement Compositions

Exemplary dietary supplement composition pre-mixes suitable for forming dietary supplement compositions of the present invention were prepared by mixing various amounts of the following components shown in Tables 8-12 below. Any of the compositions shown in Tables 8-12 may be combined with one or more ingredients disclosed above to form a dietary supplement composition of the present invention. For example, the compositions shown in Tables 8-12 may be combined with one or more of the following exemplary ingredients curcumin or a curcumin derivative (e.g., an esterified curcumin having one or more hydroxyl groups thereon converted to an ester), resveratrol or a resveratrol ester, carnitine or a carnitine derivative (e.g., L-carnitine tartrate and/or acetyl-L-carnitine), a protease complex comprising (i) papain, (ii) bromelain, or (iii) papain and bromelain, etc.

TABLE 8
Exemplary Dietary Supplement Pre-Mix Compositions

| Component | Label Name/Extract Component | Amount | |
|---|---|---|---|
| Resveratrol (Grape Seed | 5-[2-(4- | 25 | mg |
| Extract) 95% | hydroxyphenyl)ethenyl]benzene- | | |
| | 1,3-diol | | |
| Curcumin (High Potency | 1,9-Bis(4-hydroxy-3- | 100 | mg |
| Cucurmin Extract) | methoxyphenyl)-2,7- | | |
| | nonadiene-4,6-dione | | |
| resveratrol tri-acetate | | 25 | mg |
| ester | | | |
| curcumin mono-acetate | | 100 | mg |
| ester | | | |
| curcumin di-acetate ester | | 100 | mg |
| D-aspartic acid | | 3000 | mg |
| L-aspartic acid | | 250 | mg |
| n-methyl-d-aspartate | | up to 50 | mg |
| D-aspartic acid methyl | | up to 50 | mg |
| ester | | | |
| Fungal protease | | 100 | mg |
| 60,000 HUT | | | |
| bromelain | | 100 | mg |
| Hydrophilic | | 200 | mg |
| hypromellose polymers | | | |

TABLE 9
Exemplary Dietary Supplement Pre-Mix Compositions

| | Label Name/ | | |
|---|---|---|---|
| Component | Extract Component | Amount | |
| D-aspartic acid | | 3000 | mg |
| L-aspartic acid | | 250 | mg |
| n-methyl-d-aspartate | | up to 50 | mg |
| D-aspartic acid methyl | | up to 50 | mg |
| ester | | | |

TABLE 10
Exemplary Dietary Supplement Pre-Mix Compositions

| Component | Label Name/Extract Component | Amount |
|---|---|---|
| D-aspartic acid | | 3000 mg |

TABLE 11
Exemplary Dietary Supplement Pre-Mix Compositions

| Component | Label Name/Extract Component | Amount |
|---|---|---|
| n-methyl-d-aspartate | | up to 50 mg |

TABLE 12
Exemplary Dietary Supplement Pre-Mix Compositions

| | Label Name/ | | |
|---|---|---|---|
| Component | Extract Component | Amount | |
| n-methyl-d-aspartate | | up to 50 | mg |
| D-aspartic acid | | up to 500 | mg |
| Resveratrol (Grape | 5-[2-(4- | up to 50 | mg |
| Seed Extract) 95% | hydroxyphenyl)ethenyl]benzene- | | |
| | 1,3-diol | | |
| Curcumin (High | 1,9-Bis(4-hydroxy-3- | up to 150 | mg |
| Potency Cucurmin | methoxyphenyl)-2,7-nonadiene- | | |
| Extract) | 4,6-dione | | |
| resveratrol tri-acetate | | up to 50 | mg |
| ester | | | |

### Example 8

### Preparation of Exemplary Dietary Supplement Compositions

Exemplary dietary supplement compositions of the present invention were prepared by mixing the components shown in Table 2 with the components shown in Tables 10 and 11.

### Example 9

### Preparation of Exemplary Dietary Supplement Compositions

Exemplary dietary supplement compositions of the present invention were prepared by mixing the components shown in Table 3 with the components shown in Tables 10 and 11.

### Example 10

### Preparation of Exemplary Dietary Supplement Compositions

Exemplary dietary supplement compositions of the present invention were prepared by mixing various amounts of the following components shown in Table 13 below. Although not shown in Table 13, other inactive ingredients may be present including, but not limited to, dicalcium phosphate, magnesium stearate, silica, microcrystalline cellulose, hydroxypropyl methyl cellulose, hydroxypropyl cellulose, and one or more flavor-providing components. Typically, each of the additional inactive ingredients is independently present in an amount of up to about 500 mg (i.e., any amount greater than 0 up to 500 mg).

TABLE 13
Exemplary Dietary Supplement Compositions

| Component | Amount | |
|---|---|---|
| Thiamine Mononitrate (Vit B1) | 50 | mg |
| Riboflavin (Vit B2) | 50 | mg |
| Niacin (Vit B3) | 50 | mg |
| Pyridoxine HCl (Vit B6) | 50 | mg |
| Folic Acid | 0.4 | mg (400 mcg) |
| Biotin 1% | 3 | mg (30 mcg) |
| Pantothenic Acid (Vit B5) | 100 | mg |
| Vit D3 (Cholecalciferol) | 2,000 | IU |
| Cobalt Chelate 1% | 0.25 | mg |
| Portulaca Oleracea Ext. | 60 | mg |
| Arachidonic Acid | 60 | mg |
| Resveratrol 50% | up to 50 | mg |
| Curcumin 95% | 25 | mg |
| Alpha Lipoic Acid | 25 | mg |
| Acetyl-L-Carnitine | 25 | mg |
| L-Arginine Alpha Ketoglutarate | 3,000 | mg |
| L-Citrulline Malate | 100 | mg |
| Piperine Extract 95% | 5 | mg |
| Enzyme Blend* | 200 | mg |
| Cyanocobalamin.1% (Vit B12) | 10 | mg (100 mcg) |
| D-Aspartic Acid | up to 10 | mg |
| N-Methyl-D-Aspartate | up to 10 | mg |
| Resveratrol Triacetate | up to 50 | mg |
| Iron (Ferrous Gluconate) | 2 | mg |

*Enzyme Blend comprises a combination of bromelain, cellulase, amylase, protease, lipase, and papain.

### Example 11

### Preparation of Exemplary Dietary Supplement Compositions

Exemplary dietary supplement compositions of the present invention were prepared by mixing various amounts of the following components shown in Table 14 below. Although not shown in Table 14, other inactive ingredients may be present including, but not limited to, dicalcium phosphate, magnesium stearate, and one or more flavor-providing components. Typically, each of the additional inactive ingredients is independently present in an amount of up to about 500 mg (i.e., any amount greater than 0 up to 500 mg).

TABLE 14
Exemplary Dietary Supplement Compositions

| Component | Amount | |
|---|---|---|
| Resveratrol 98% | 25 | mg |
| Curcumin 95% | 25 | mg |
| Tong Kat Ali Ext. (100:1) | 25 | mg |
| Mucuna pruriens Ext. (25% L-Dopa) | 25 | mg |
| Bioperine ™ extract | 5 | mg |
| S-adenosyl methionine (SAMe) | 1 | mg |
| Trimethylglycine (Betaine Anhydrous) | 9 | mg |
| D-Aspartic Acid | up to 25 | mg |
| N-Methyl-D-Aspartate | up to 25 | mg |
| Resveratrol Triacetate | up to 25 | mg |
| Magnesium Hydroxide | 84 | mg |
| Zinc Citrate 32% | 4.68 | mg |

### Example 12

### Preparation of Exemplary Dietary Supplement Compositions

Exemplary dietary supplement compositions of the present invention were prepared by mixing various amounts of the following components shown in Table 15 below. Although not shown in Table 15, other inactive ingredients may be present including, but not limited to, N&A Flavors, Acesulfame K, Sucralose, Citric Acid, and Natural Color. Typically, each of the additional inactive ingredients is independently present in an amount of up to about 500 mg (i.e., any amount greater than 0 up to 500 mg).

TABLE 15
Exemplary Dietary Supplement Compositions

| Component | Amount | |
|---|---|---|
| Caffeine Anhydrous | up to 600 | mg |
| 1,3-Dimethylamilamine | up to 200 | mg |
| Taurine | up to 4000 | mg |
| D-Glucuronolactone | 200 | mg |
| Resveratrol 50% | up to 25 | mg |
| Curcumin (Turmeric Root 4:1) | 100 | mg |
| Alpha Lipoic Acid | 25 | mg |
| Acetyl-L-Carnitine | 25 | mg |
| Evodiamine Extract 98% | 0.1 | mg |
| L-Arginine Orotate 2:1 | 900 | mg |
| Beta Alanine | 50 | mg |
| Trimethylglycine (Betaine Anhydrous) | 50 | mg |
| L-Leucine | 500 | mg |
| L-Isoleucine | 250 | mg |
| L-Valine | 250 | mg |
| Piperine Extract 95% | 5 | mg |
| Enzyme Blend EB13009-01* | 200 | mg |
| Glycerol Monostearate | 950 | mg |
| D-Aspartic Acid | up to 10 | mg |
| N-Methyl D-Aspartate | up to 10 | mg |
| Resveratol Triacetate | up to 25 | mg |

*Enzyme Blend comprises a combination of bromelain, cellulase, amylase, protease, lipase, and papain.

### Example 13

### Preparation of Exemplary Dietary Supplement Compositions

Exemplary dietary supplement compositions of the present invention were prepared by mixing various amounts of the following components shown in Table 16 below. Although not shown in Table 16, other inactive ingredients may be present including, but not limited to, N&A Flavors, Acesulfame K, Sucralose, Citric Acid, and Natural Color. Typically, each of the additional inactive ingredients is independently present in an amount of up to about 500 mg (i.e., any amount greater than 0 up to 500 mg).

TABLE 16
Exemplary Dietary Supplement Compositions

| Component | Amount | |
|---|---|---|
| Resveratrol 50% | up to 25 | mg |
| Curcumin (Turmeric Root 4:1) | 100 | mg |
| Alpha Lipoic Acid | 25 | mg |
| Acetyl-L-Carnitine | 25 | mg |
| L-Glutamine | 500 | mg |
| L-Arginine | 500 | mg |
| L-Leucine | 2000 | mg |
| L-Isoleucine | 250 | mg |
| L-Valine | 250 | mg |
| L-Lysine | 500 | mg |
| L-Phenylalanine | 500 | mg |
| L-Threonine | 500 | mg |
| L-Histidine | 250 | mg |
| L-Methionine | 250 | mg |
| Waxy Maize Starch | 2000 | mg |
| Maltodextrin | 2000 | mg |
| Piperine Extract 95% | 5 | mg |
| Enzyme Blend EB13009-01* | 200 | mg |
| Glycerol Monostearate | 1000 | mg |
| D-Aspartic Acid | up to 10 | mg |
| N-Methyl D-Aspartate | up to 10 | mg |
| Resveratol Triacetate | up to 25 | mg |

*Enzyme Blend comprises a combination of bromelain, cellulase, amylase, protease, lipase, and papain.

### Example 14

### Preparation of Exemplary Dietary Supplement Compositions

Exemplary dietary supplement compositions of the present invention were prepared by mixing various amounts of the following components shown in Table 17 below. Although not shown in Table 17, other inactive ingredients may be present including, but not limited to, N&A Grape Flavor, Acesulfame K, Sucralose, Citric Acid, and Natural Color. Typically, each of the additional inactive ingredients is independently present in an amount of up to about 500 mg (i.e., any amount greater than 0 up to 500 mg).

TABLE 17
Exemplary Dietary Supplement Compositions

| | |
|---|---|
| Component | Amount |

| | | |
|---|---|---|
| Caffeine Anhydrous | up to 600 | mg |
| 1,3-Dimethylamilamine | up to 200 | mg |
| Taurine | up to 4000 | mg |
| D-Glucuronolactone | 200 | mg |
| Resveratrol 50% | up to 25 | mg |
| Curcumin (Turmeric Root 4:1) | 100 | mg |
| Alpha Lipoic Acid | 100 | mg |
| Acetyl-L-Carnitine | 25 | mg |
| Evodiamine Extract 98% | 0.1 | mg |
| L-Arginine Orotate 2:1 | 900 | mg |
| Beta Alanine | 50 | mg |
| Trimethylglycine (Betaine Anhydrous) | 50 | mg |
| L-Leucine | 500 | mg |
| L-Isoleucine | 250 | mg |
| L-Valine | 250 | mg |
| Piperine Extract 95% | 5 | mg |
| Enzyme Blend EB13009-01* | 200 | mg |
| Glycerol Monostearate | 900 | mg |
| D-Aspartic Acid | up to 10 | mg |
| N-Methyl D-Aspartate | up to 10 | mg |
| Resveratol Triacetate | up to 25 | mg |

*Enzyme Blend comprises a combination of bromelain, cellulase, amylase, protease, lipase, and papain.

### Example 15

### Preparation of Exemplary Dietary Supplement Compositions

Exemplary dietary supplement compositions of the present invention were prepared by mixing various amounts of the following components shown in Table 18 below. Although not shown in Table 18, other inactive ingredients may be present including, but not limited to, N&A Grape Flavor, Acesulfame K, Sucralose, Citric Acid, and Natural Color. Typically, each of the additional inactive ingredients is independently present in an amount of up to about 500 mg (i.e., any amount greater than 0 up to 500 mg).

TABLE 18
Exemplary Dietary Supplement Compositions

| Component | Amount | |
|---|---|---|
| Caffeine Anhydrous | up to 600 | mg |
| 1,3-Dimethylamylamine HCL | up to 800 | mg |
| Synephrine HCL | 5 | mg |
| B-Phenylethylamine HCL | 5 | mg |
| Curcumin 95% | 5 | mg |
| Resveratrol 98% | 5 | mg |
| Bioperine ™ extract | 2.5 | mg |
| Taurine | up to 1000 | mg |
| Banaba Leaf Extract 1% | 0.5 | mg |
| Cinnamon Leaf Extract | 0.5 | mg |
| Irvignia Gabonesis Seed PE 4:1 | 100 | mg |
| Evodiamine 98% | 0.5 | mg |
| Gugglesterones 95% E and Z | 0.5 | mg |
| Quercetin Dihydrate | 0.5 | mg |
| Ashwagandha Extract (Withaferin A) | 0.5 | mg |
| Green Tea Extract EGCG 70% | 0.5 | mg |
| Syloid 244 | 3 | mg |
| Magnesium Stearate | 1.75 | mg |
| Vitamin D-3 100,000 IU | 10 | mg |
| Genistein 80% | 0.5 | mg |
| Iodine 5% | 0.25 | mg |
| 3,5-Diiodo-L-Thyronine | up to 25 | mg |
| 3,3-Diiodo-L-Thyronine | up to 25 | mg |
| Sodium Selenate 0.2% Trit | 5 | mg |

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A dietary supplement composition comprising:
at least one component selected from curcumin and a curcumin derivative; and
at least one component selected from resveratrol and a resveratrol ester.

2. The dietary supplement composition of claim 1, wherein said composition comprises curcumin.

3. The dietary supplement composition of claim 2, wherein said composition comprises a resveratrol ester.

4. The dietary supplement composition of claim 3, wherein said resveratrol ester comprises resveratrol triacetate.

5. The dietary supplement composition of claim 4, wherein said composition further comprises resveratrol.

6. The dietary supplement composition of claim 5, wherein said composition further comprises carnitine or a carnitine derivative.

7. The dietary supplement composition of claim 6, wherein said composition further comprises a carnitine derivative comprising (i) L-carnitine tartrate, (ii) acetyl-L-carnitine, (iii) L-carnitine tartrate and acetyl-L-carnitine, or (iv) L-carnitine fumarate.

8. The dietary supplement composition of claim 5, wherein said composition further comprises a protease complex comprising (i) papain, (ii) bromelain, or (iii) papain and bromelain.

9. The dietary supplement composition of claim 5, wherein said composition further comprises L-leucine, L-isoleucine and L-valine.

10. The dietary supplement composition of claim 9, wherein said composition further comprises taurine.

11. The dietary supplement composition of claim 10, wherein said composition further comprises evodiamine.

12. The dietary supplement composition of claim 11, wherein said composition further comprises 1,3-dimethylamylamine HCL.

13. The dietary supplement composition of claim 7, wherein said composition further comprises alpha lipoic acid.

14. The dietary supplement composition of claim 13, wherein said composition further comprises L-arginine alpha ketoglutarate.

15. A method of supplementing a diet of an animal, said method comprising:
ingesting the dietary supplement composition of claim 1.

16. A method of supplementing a diet of an animal, said method comprising:
ingesting the dietary supplement composition of claim 5.

17. A method of supplementing a diet of an animal, said method comprising:
ingesting the dietary supplement composition of claim 13.

18. A method of providing one or more benefits to a human, said method comprising:
ingesting a dietary supplement composition comprising:
at least one component selected from curcumin and a curcumin derivative; and
at least one component selected from resveratrol and a resveratrol ester;
wherein the one or more benefits comprise one or more of: heat shock response stimulation for muscle growth; enhance proper protein folding and apoptosis; muscle repair; muscle recuperation from training; recovery from trauma, injury or intense exercise; speed muscle growth; pharmacologically inhibit the transcription factor NF-kappaB to enhance muscle regeneration; reduce inflammation; provide anti-aging benefits; modulate estrogen receptor; increase testosterone; and stimulate increases in hormone levels.

19. The method of claim 18, wherein the dietary supplement composition comprises curcumin and a resveratrol ester.

20. The method of claim 19, wherein the resveratrol ester comprises resveratrol triacetate.
